(19) United States
(12) Patent Application Publication
(10) Pub. No.: US 2025/0181815 A1
(43) Pub. Date: Jun. 5, 2025

(54) METHOD OF SIMULATING THE BEHAVIOUR OF AN ELECTRONIC DEVICE

(71) Applicant: NANOACADEMIC TECHNOLOGIES INC., Montréal (CA)

(72) Inventors: Pericles PHILIPPOPOULOS, Montréal (CA); Felix BEAUDOIN, Montréal (CA)

(21) Appl. No.: 18/855,047

(22) PCT Filed: May 15, 2023
(86) PCT No.: PCT/CA2023/050664

§ 371 Date: Oct. 8, 2024

Related U.S. Application Data
(60) Provisional application No. 63/342,278, filed on May 16, 2022.

(51) Int. Cl. G06F 30/398; G06F 30/392; G16C 10/00; G16C 20/30

(52) U.S. Cl. CPC G06F 30/398; G06F 30/392; G16C 10/00; G16C 20/30

(57) ABSTRACT

A computer-implemented method of simulating behaviour of an electronic device, comprising: providing a mesh having nodes spatially distributed across a geometrical model of the electronic device; calculating, taking into consideration the boundary conditions imposed to some of the nodes, and for each of the remaining ones of the nodes, i) current values of electric potential using a linearization of a non-linear Poisson equation, and ii) current values of charge density using a charge density model, determining an error between the starting values and the current values, when the error is not within an error threshold, changing the spatial distribution of the nodes to a current spatial distribution of nodes, and repeating said steps of calculating and determining with the recent values and mesh; and when the error is within an error threshold, outputting the current values of electric potential and charge density as a simulated behaviour of the electronic device.

### BACKGROUND

Several problems in physics can be described by a Poisson equation of the general form ∇·(ε∇ϕ)=−ρ. For instance, the design of solid-state electronic devices can heavily rely on simulating the behaviour of a proposed design under different sets of conditions, before launching fabrication, and simulating the behaviour of such devices can rely on solving a Poisson's equation. In many cases, the problem may be better represented by a non-linear form of the Poisson equation, given by

$$∇ ( ε ∇ ϕ ) = - e ( p - n + {N}_{+} - {N}_{-} ) = - ρ , \qquad ( 1 )$$

where ε is the dielectric permittivity of the medium, ϕ is the electric potential, e is the elementary charge (e>0), p and n are the hole and electron densities, respectively, and N₊ and N₋ are the densities of ionized donors and acceptors, respectively. When ρ depends on the variable ϕ for which the equation is solved through at least one variable among p, n, N₊, and N₋, the Poisson equation can be said to be non-linear. One approach to solve non-linear Poisson equations is to apply an iterative method using a computer. In an iterative method, calculations are repeated over multiple instances and inputs to the calculations are adjusted based on the results of one or more previous instances. The equation is considered “solved” when the results “converge”. Convergence typically involves determining that the error is below a given threshold associated to a level of accuracy deemed satisfactory for the circumstances.

### SUMMARY

While various computer-implemented methods of solving Poisson equations exist, there remains room for improvement. For instance, quantum electronic devices, such as spin qubits in semiconductors are not only difficult to fabricate but also often display unwanted behavior once fabricated. While former computer-aided design (TCAD) software can be used to predict the electrostatics of classical (non-quantum) semiconductor devices, such tools lacked two main features in order to reliably calculate (predict) spin-qubit performance metrics before fabrication: (i) the capability to account for quantum-mechanical effects like quantum confinement, quantum superpositions, and entanglement, which are not mere corrections to classical TCAD models but become operating principles of spin qubits, and (ii) convergence of non-linear Poisson solvers at cryogenic temperatures at which spin qubits are operated (computer-implemented methods can begin to fail to converge more and more frequently as the temperature of the model goes below 100 K).

The detailed description presented below describes a computer-implemented method of solving a non-linear Poisson equation. The method can allow reliable convergence below 100K, and in some cases, even at deep cryogenic temperatures (e.g. 10 mK-4K). The method can be included, for instance, in the context of a computer-aided design software including quantum-mechanical solvers and used to predict spin-qubit. The algorithm may thus open the door to robust, high-throughput computer-aided design of spin qubits operated in realistic experimental and industrial settings, to name one possible example. This algorithm can also potentially be used for computer-aided design of other quantum systems such as superconducting qubits.

In accordance with one aspect, there is provided: a computer-implemented method of simulating the behaviour of an electronic device in the context of given boundary conditions, the behavior including a charge density distribution and an electric potential distribution over a geometry of the electronic device, the method comprising: providing a mesh having nodes spatially distributed across a geometrical model of the electronic device; calculating, taking into consideration the boundary conditions imposed to some of the nodes, and for each of the remaining ones of the nodes, i) current values of electric potential using a linearization of a non-linear Poisson equation, and ii) current values of charge density using a charge density model, wherein the current values of a first one of electric potential and of charge density are calculated further based on starting values of a second one of electric potential and of charge density, and the current values of the second one is calculated further based on the current values of the first one; determining an error between the starting values and the current values; when the error is not within an error threshold, changing the spatial distribution of the nodes to a current spatial distribution of nodes, and repeating said steps of calculating and determining i) using the current values previously calculated as the starting values and ii) using the current spatial distribution of nodes for the nodes; and when the error is within an error threshold, outputting the current values of electric potential and charge density as a simulated behaviour of the electronic device.

In accordance with another aspect, there is provided: a computer implemented method of determining the behaviour of an electronic device in predetermined conditions, the behavior including a charge density distribution and an electric potential distribution over a geometry of the electronic device, the method comprising: providing an initial mesh having nodes distributed across a geometric model of the electronic device, each node having an initial spatial coordinates corresponding to a location forming part of the geometric model; providing a charge density model; provide a non-linear Poisson equation of the form ∇(ε∇ϕ)=−ρ, where ε is the dielectric permittivity of each one of the nodes, ϕ is the electric potential of each one of the nodes, and ρ is the charge density of each one of the nodes, and where both the electric potential and the charge density are i) both a priori unknown and ii) related to one another based on the charge density model; providing a linearization of the non-linear Poisson equation; providing boundary conditions having set values of charge density and electric potential for some of the nodes; providing an initial values of a first one of electric potential and charge density for remaining ones of the nodes; providing an error threshold; calculating a values of the second one of electric potential and charge density for the remaining ones of the nodes; calculating a subsequent values of the first one of electric potential and charge density for remaining ones of the nodes; wherein said calculating values for charge density includes using the charge density model, and calculating values for the electric potential uses the linearization of the non-linear Poisson equation; calculate an error between the initial values and the subsequent values; when the error is within the threshold, outputting the latest calculated values of electric potential and charge density; and when the error is not within the threshold, generating a subsequent mesh with a different spatial distribution of nodes than the distribution of nodes of the initial mesh, and repeating the steps of calculating using the latest calculated value of electric potential or charge density as the initial value of electric potential and charge density.

In accordance with one aspect, there is provided: a computer-implemented method of simulating the behaviour of a composition of matter in the context of given boundary conditions, the behavior including a charge density distribution and an electric potential distribution over a geometry of the composition of matter, the method comprising: providing a mesh having nodes spatially distributed across a geometrical model of the composition of matter; calculating, taking into consideration the boundary conditions imposed to some of the nodes, and for each of the remaining ones of the nodes, i) current values of electric potential using a linearization of a non-linear Poisson equation, and ii) current values of charge density using a charge density model, wherein the current values of a first one of electric potential and of charge density are calculated further based on starting values of a second one of electric potential and of charge density, and the current values of the second one is calculated further based on the current values of the first one; determining an error between the starting values and the current values; when the error is not within an error threshold, changing the spatial distribution of the nodes to a current spatial distribution of nodes, and repeating said steps of calculating and determining i) using the current values previously calculated as the starting values and ii) using the current spatial distribution of nodes for the nodes; and when the error is within an error threshold, outputting the current values of electric potential and charge density as a simulated behaviour of the composition of matter.

Many further features and combinations thereof concerning the present improvements will appear to those skilled in the art following a reading of the instant disclosure.

### DESCRIPTION OF THE FIGURES

In the figures,

FIG. 1 is a flowchart of a computer-implemented method of solving a non-linear Poisson equation in accordance with a first embodiment;

FIG. 2 is an oblique view of a mesh modeling an electronic device;

FIG. 3 is a flowchart of a computer-implemented method of solving a non-linear Poisson equation in accordance with a second embodiment;

FIG. 4 is a flow chart of an example of a mesh adapter module;

FIG. 5 is a flowchart of a computer-implemented method of solving a non-linear Poisson equation in accordance with a third embodiment;

FIG. 6 is a flow chart representing one possible example of a software package incorporating a computer-implemented method of solving a non-linear Poisson equation;

FIG. 7: Computer-aided design (CAD) model and automatically generated mesh. (a) 3D Ultra-Thin Body and Buried oxide (UTBB) Fully-Depleted Silicon-On-Insulator (FD-SOI) geometry. (b) Slice along the dash-dotted orange line in (a). (c) Slice along the dotted green line in (a). Gray volumes: silicon dioxide. Dark green volumes: n-doped silicon source and drain (doping density 10²¹ cm⁻³). Pale green volumes: undoped silicon channel. Dark blue surfaces: source and drain Ohmic contacts. Red surfaces: gate boundaries with labels FgB and FgT (bottom and top front gates, respectively), and G1, G2, G3 (side gates 1, 2, and 3). Magenta lines: bottom surface Ohmic boundary. Dashed pale blue lines: delimitation of the quantum-dot region in which Schrödinger's equation is solved. Yellow rectangle: approximate location at which a quantum dot is formed. The mesh shown here was produced using Gmsh, and obtained using QTCAD's adaptive technique leading to robust convergence of the non-linear Poisson solver at 100 mK with 953,141 global nodes.

FIG. 8 Poisson and Schrödinger simulations of a 28 nm UTBB FD-SOI device with QTCAD. (a) Conduction band edge E_{c} throughout the device. The first color scale and the corresponding density plot represents E_{c} on an x·y plane lying 1 Å below the interface between the silicon channel and the oxide that separates it from the gates FgT, FgB and G2. The second color scale corresponds to the 3D equipotential isosurfaces in the vicinity of G3. The white rectangles in front of G3 delimit the slices over which density plots of the single-electron envelope functions are plotted. The bias V_{G} on each gate G is: V_{FgT}=V_{FgB}=0.843 V, V_{G1}=0. V, V_{G2}=1.7 V, and V_{G3}=4 V (no bias is applied on the back gate). (b) Density plot of the single-electron ground state over a slice within a x·z plane [white rectangle parallel to G3 in (a)]. (c) First excited state over a slice within a y·z plane [white rectangle perpendicular to G3 in (a)](d) Second excited state over the x·z slice. In (b-d), the white lines illustrate the interface between silicon dioxide and the silicon channel hosting the electron envelope functions. (e) First three eigenenergies as a function of G3 bias. Black dot: ground state. Red triangle: first excited state. Blue cross: second excited state. Solid green line: linear fit giving the lever arm of G3 over the quantum-dot energy levels. Dashed magenta line: Fermi level.

FIG. 9 is a table summarizing simulation outcomes for 10 random bias configurations. V_{FgB}, V_{FgT}, V_{G1}, V_{G2}, V_{G3}, and V_{B} give the gate biases in volts;

FIG. 10 is a block diagram representing a computer.

### DETAILED DESCRIPTION

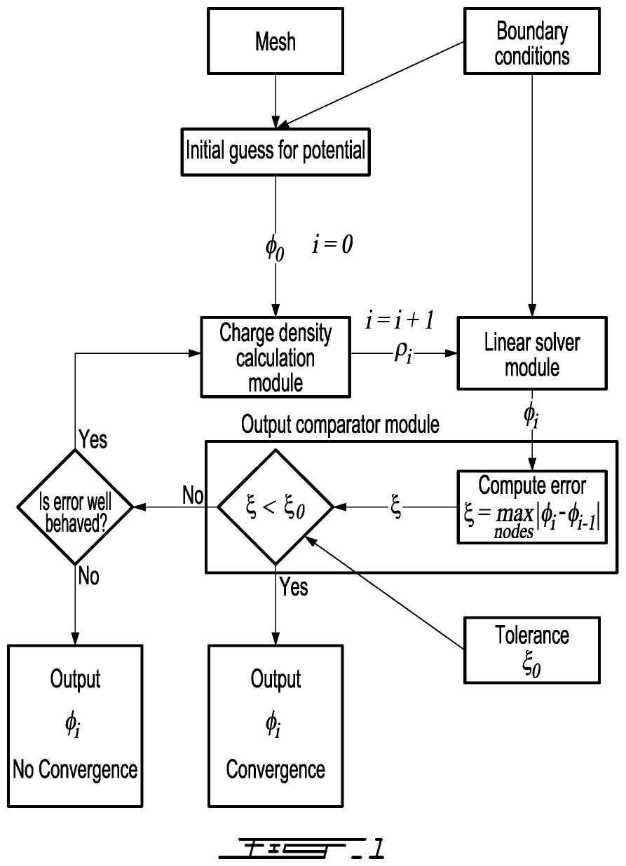

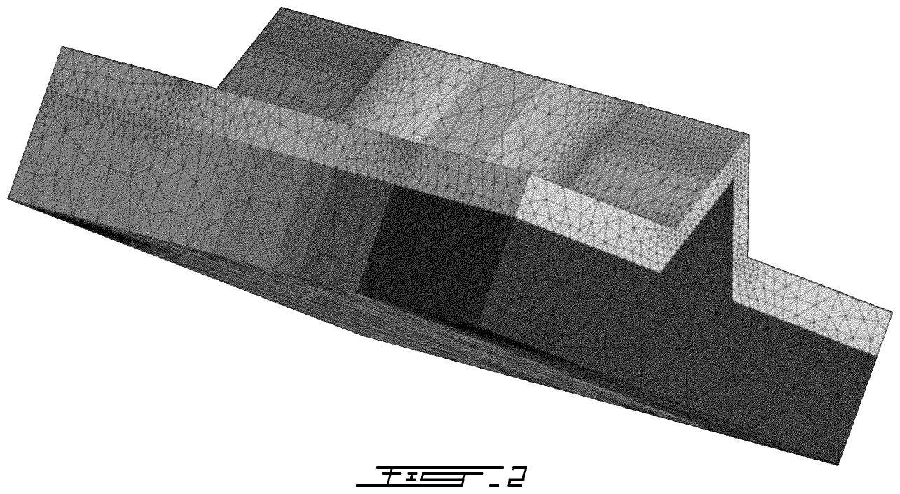

FIG. 1 presents a flow chart outlining a first example of a computer-implemented method of solving a non-linear Poisson's equation. The method can alternately be referred to as a “Poisson Solver” for simplicity. As will be presented further below, such a method can be implemented as part of a more general software application configured for simulating the behaviour of an electronic device, such as a device relying on electrons, holes (possibly opto-electronic, and/or including quantum effects), or simulating the electrostatics of a different composition of matter (e.g. ionic solutions, other charged particles . . . ).

In this first example, the method receives three inputs: i) a mesh and ii) a set of boundary conditions that define the problem we are solving, and iii) a threshold, or tolerance ξ₀, which represents the degree of accuracy which is considered satisfactory in a given context to consider that the method has converged. In an alternate embodiment, the threshold ξ₀ can be predetermined or otherwise not received as an input.

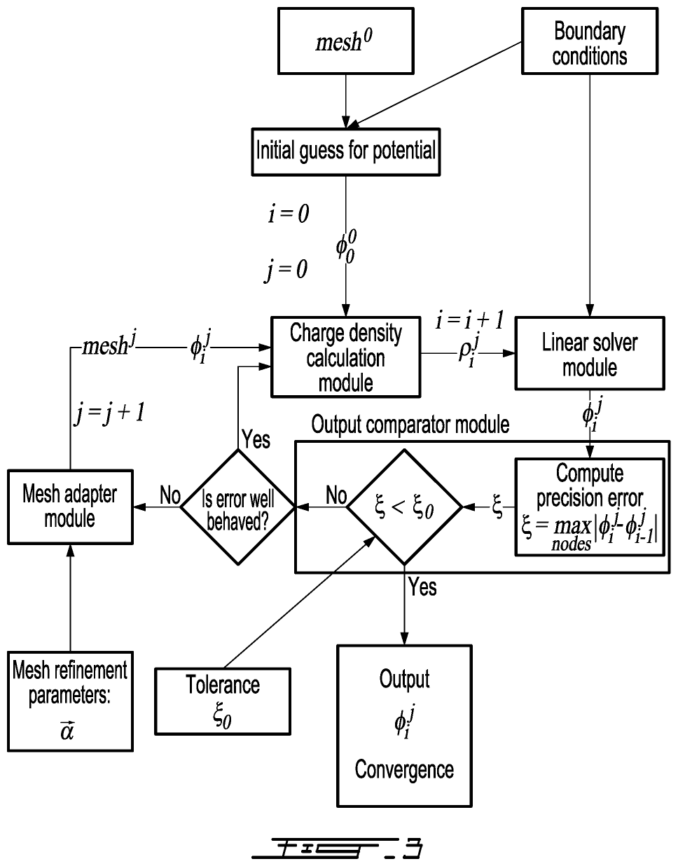

There are various methods of generating a suitable mesh. These various methods can be said to lead to a distribution of nodes across the volume of a geometrical model of the electronic device, or other composition of matter, whose behaviour is being simulated. An example mesh is presented in FIG. 2, for instance, where all intersections between black lines represent nodes. In a non-linear Poisson's equation, the electric potential ϕ and charge density ρ are i) both a priori unknown and ii) related to one another based on a relationship defined by a “charge density model”. The conditions for which the behaviour is to be simulated can be defined as “boundary conditions” in the form of imposed solutions or values of electric potential ϕ and charge density ρ for a subset of the nodes, typically nodes associated with one or more surface of the geometrical model. Simulating the charge density distribution and electric potential distribution over the geometry can involve solving ϕ and ρ for each one of the remaining nodes nodes, i.e. the nodes for which no value or solution is imposed by the boundary conditions.

Referring back to FIG. 1, the method generally includes a charge density calculation module, configured for calculating values of charge density ρ based on provided values of electric potential ϕ (e.g. values calculated by a previous iteration or provided as an initial “guess”), using the charge density model. The values of charge density ρ outputted by the charge density calculation module are then communicated to a linear solver module. The linear solver module is configured for calculating values of electric potential ϕ based on the values of charge density ρ using a linearisation of the non-linear Poisson equation. However, it will be understood that in alternate embodiments, the linear solver module can operate before the charge density calculation module rather than after, i.e. their positions can be interchanged in the flow chart of FIG. 1.

Henceforth, in the illustrated embodiment, the method is configured here for solving for ϕ, though it can alternately be configured for solving for ρ, such as by switching the positions of the charge density calculation module and of the linear solver module. In several embodiments, the boundary conditions can be used to generate an initial guess for the solution, ϕ₀, over the mesh, but in some alternate embodiments, an initial guess for the solution, ϕ₀, can be provided as an input regardless of boundary conditions or calculated in any suitable manner. In an embodiment where the method is configured for solving for ρ, an initial guess can be provided for ρ rather than for ϕ₀, as will be understood by persons having ordinary skill in the art. The generation of the initial guess can be considered to be a step of the method. After generating ϕ₀, a counter represented here by the variable i can be initialized to i=0 and incremented over subsequented iterations of the method to help keep track of the steps of the procedure. A next step can be referred to as running a charge density calculation module (the expression “module” is used herein to referto a software function), which is tasked with computing the associated charge density.

Depending on the embodiment, the charge density calculation module can work in different ways. For example, referring to a Poisson's equation phrased such as in equation (1) above, it can compute the charge density, ρᵢ, as a function of ϕᵢ₋₁, i.e. ρᵢ=ρ(ϕᵢ₋₁). In this embodiment, a next step can be referred to as running a linear solver module. The linear solver module can, for instance, be tasked with solving for ϕ in Eq. 1 given the specified boundary conditions, and assuming the right hand side is fixed and given by

$$e ( p - n + {N}_{+} - {N}_{-} ) = {ρ}_{i} . \qquad ( 2 )$$

Alternatively, the charge density calculation module can be configured to compute ρᵢ by linearizing the functional ρ(ϕ) according to Newton's method. In this case we have

$${ρ}_{i} = ρ ( {ϕ}_{i - 1} ) + {ρ}^{′} ( {ϕ}_{i - 1} ) ( {ϕ}_{i} - {ϕ}_{i - 1} ) , \qquad ( 3 )$$

where ρ′ is the functional derivative of ρ(ϕ). In this case, using equation 1 where we assume that the right hand side is given by equation 3 we have

$$∇ \cdot ( ε ∇ ϕ i ) + {ρ}^{′} ( {ϕ}_{i - 1} ) ( {ϕ}_{i} - {ϕ}_{i - 1} ) = - ρ ( {ϕ}_{i - 1} ) , \qquad ( 4 )$$

In both cases, the linear solver module can be said to operate on a linearisation of the non-linear Poisson's equation to obtain ϕᵢ. Accordingly, there can be various charge density models used as a basis of calculation by the charge density calculation model, and various linearisation schemes of the non-linear Poisson's equation, and a suitable charge density model and a suitable linearisation of the non-linear Poisson's equation can be selected by a person having ordinary skill in the art based on the particularities of the problem which is to be addressed, and any other particularities of the context of the given embodiment.

It will be understood that the function ρ(ϕ) is based on a physical model and can be computed in a variety of different ways (e.g. assuming classical charges occupying parabolic bands, or quantum charges based on the confinement potential associated with ϕᵢ). Specific examples of such models will be given below.

The output ϕᵢ, from the linear solver module, can then be processed by an output comparator module. The output comparator module can compare the output ϕᵢ to the potential from the previous iteration ϕᵢ₋₁ to generate a precision error ξ. In one embodiment, this error can be defined as the maximal (relative or absolute) difference of ϕᵢ and ϕᵢ₋₁ over all nodes of the mesh, for instance, in accordance with

$$ξ = {max}_{nodes} \vert {ϕ}_{i} - {ϕ}_{i - 1} \vert . \qquad ( 5 )$$

Alternately, the precision error can be defined in different ways, such as a matrix for instance. If the precision error, ξ, is below (within) a user-defined threshold, ξ₀, the output comparator module can output ϕᵢ as a converged result, e.g. as a successfully simulated behaviour of an electronic device. In an embodiment, the error associated to the current values (of ϕᵢ or ρᵢ depending which is calculated first) can be a precision error in the sense that it can measure how close the values calculated for a given iteration are to the corresponding values calculated in an earlier iteration (or, in some embodiments, more than one earlier iteration). This is different from an accuracy error, for instance, which would rather provide an indication of how close or far calculated values are from a true solution of the physical problem.

The computer-implemented method can then proceed with another iteration through charge density calculation module, linear solver module, and output comparator module, until certain conditions are met, such as determining that the error has reached the stage where it is within the threshold for instance.

In some cases, it can be preferred to automatically stop the iterations when certain conditions are met. Such conditions can involve a certain maximum number of iterations for instance. In the embodiment illustrated, such conditions can include analysing for trends in the error over a number of iterations. Indeed, if the error is determined to increases consistently, or in a zig-zagging manner, over a number of iterations, the method can be determined to diverge. If the error is seen to become constant over a number of iterations, or to zig-zag while remaining constant on average over a number of iterations, the error can be determined to have saturated. In both these latter scenarios, the method can be determined to not converge, and the iterations can be stopped. The next iteration proceeding contingent upon not finding an absence of convergence, for instance.

In this embodiment, a further module is provided, which will be referred to here as an error analysis module. The error analysis module can be used to make a determination of the nature of the error. For instance, the error analysis module can determine that no convergence will be reached, and therefore trigger the end of the iterations (potentially together with a failure notification), if it is determined that the error that has saturated or that the error is diverging. Saturation can be defined as a state where the last N errors generated have all been within x % of each other (N and x can be user defined), for instance. Divergence of the error can be detected by checking that the last N errors are above some maximal threshold, Φ̅, and that they are all increasing with i (N and Φ̅ can be user defined), for instance. The event of non-convergence can also involve outputting ϕᵢ with a caveat that we have not obtained a solution to within the requested tolerance, ξ₀, i.e. the method has failed to converge. Such a non-convergence result can be referred to as a determination that the error is not “well behaved”, and its alternative, e.g. that the error is not diverging and has not saturated, can be referred to as a determination that the error is “well behaved” and lead to the next iteration of the steps outlined so far. In the next iteration of the method, ϕᵢ can be used in the charge density calculation module, i.e. the charge density calculation module can be used on the basis of ϕᵢ being the new “guess”, which then yields ρᵢ₊ₗ, and so forth for a next iteration.

It will be understood that as various linearisations of the non-linear Poisson equation can be used by the linear solver module, depending on the embodiment, various charge density models can be used by the charge density calculation module. For the purpose of providing a full and detailed specification, several examples of charge density models will now be presented, via which charge densities can be calculated from the potential, though it will be understood that this list is by no means exhaustive and that it is within the realm of the capabilities of a person having ordinary skill in the art to select a charge density model which is adapted to the specifities of the embodiment which is to be addressed.

A first category of charge density models are the electron and hole charge density models. There are potentially infinitely many ways in which electron and hole densities may be modelled in a non-linear Poisson solver. Two main approaches are presented here detail, and additional examples are presented below.

A first main approach is the charge density for classical electron and hole gases. In a bulk semiconductor at equilibrium, assuming isotropic and parabolic bands, the electron and hole densities are given by

$$n = {N}_{c} {F}_{\frac{1}{2}} ( \frac{{E}_{F} - {E}_{C}}{{k}_{B} T} ) , p = {N}_{v} {F}_{\frac{1}{2}} ( \frac{{E}_{V} - {E}_{F}}{{k}_{B} T} ) , \qquad ( 6 )$$

where E_{C} and E_{V} are the conduction and valence band edges, respectively, E_{F} is the Fermi level, T is the device temperature (assumed to be uniform), k_{B} is the Boltzmann constant, and

$${F}_{\frac{1}{2}} ( x )$$

is the complete Fermi-Dirac integral of order ½, which is explicitly given by

$${F}_{\frac{1}{2}} ( x ) = \frac{2}{\sqrt{π}} {\int }_{0}^{∞} d t \frac{\sqrt{t}}{1 + {e}^{t - x}} . \qquad ( 7 )$$

In Eq. 6, the conduction and valence band edges are determined by the electric potential ϕ. For example, under Anderson's rule, these band edges are given by

$${E}_{C} = - e ( ϕ - {ϕ}_{F} ) - χ , \qquad ( 8 )$$

$${E}_{V} = - e ( ϕ - {ϕ}_{F} ) - χ - {E}_{g} , \qquad ( 9 )$$

where e is the elementary (positive) electric charge, λ is the electron affinity, E_{g} is the band-gap energy of the semiconductor, and ϕ_{F} is an arbitrary reference potential. Finally, in Eq. 6, we have introduced the electron and hole effective densities of states

$${N}_{c} = \frac{{g}_{c}}{8} {( \frac{2 {k}_{B} T {m}_{c}}{π {ℏ}^{2}} )}^{3 / 2} , {N}_{v} = \frac{{g}_{ν}}{8} {( \frac{2 {k}_{B} T {m}_{ν}}{π {ℏ}^{2}} )}^{3 / 2} , \qquad ( 10 )$$

where g_{c(v)} is the total electron (hole) band degeneracy (including both spin and valley degeneracies), ℏ is the reduced Planck constant, and m_{c(v)} is the electron (hole) density-of-states effective mass.

Usage of these electron and hole density expressions within a non-linear Poisson solver is sometimes referred to as the Thomas-Fermi approximation.

A second main approach is the charge density for quantum-confined electrons and holes. For electrons or holes occupying a nanostructure, with strong confinement along 1, 2, or 3 dimensions, the classical model outlined above may not provide an accurate description of the charge density. Instead, a more accurate description may be obtained by considering a quantum-mechanical model. Such a model is characterized by quantized energy levels (or subbands) of the confined particles with energies Eᵥ (v being an index that labels the levels or subbands).

In a single-band model, the carrier density for electrons confined along three dimensions, can be computed using

$$n ( r ) = {g}_{q , e} {Σ}_{v} {n}_{F} ( \frac{{E}_{ν} - {E}_{F}}{{k}_{B} T} ) {\vert {F}_{ν} ( r ) \vert}^{2} , \qquad ( 11 )$$

where n_{F}(x)≡1/(1+e^{x}) is the Fermi-Dirac distribution, Fᵥ(r) is the v-th quantized energy eigenfunction of the nanostructure, and g_{q,e} is the total (spin and valley) degeneracy of the quantized energy levels.

Eq. 11 can be generalized for multi-band models, which are often reserved for holes. In such cases, the carrier density is given by

$$p ( r ) = {Σ}_{ν} {n}_{F} ( \frac{{E}_{ν} - {E}_{F}}{{k}_{B} T} ) {Σ}_{n} {\vert {F}_{n , ν} ( r ) \vert}^{2} , \qquad ( 12 )$$

where F_{n,v}(r) is the projection on band n of the envelope function of the v-th eigensolution of the coupled envelope function equations with energy Eᵥ. Note that unlike Eq. 11, Eq. 12 does not have a degeneracy factor; possible degeneracies are instead considered through the sum over v.

Similar expressions exist for both electrons and holes when considering 1- or 2-dimensional confinement.

Usage of quantum-confined electron or hole densities such as those displayed above within a non-linear Poisson solver is conventionally called the Schrödinger-Poisson approach.

Several other approaches can be used for electron and hole density. We discuss some of them below. One additional approach is via density-functional theory (DFT). In this approach, an effective Schrödinger equation is solved where the Hamiltonian contains both a term that depends on the electric potential and an exchange-correlation term that depends on the charge density. The effective Schrödinger equation and the eigenfuntions of the Hamiltonian are often called Kohn-Sham equation and Kohn-Sham orbitals, respectively. Eqs. (11) and (12) can be used to compute the charge density by replacing the envelope functions, F_{n,v}(r) and Fᵥ(r), by the Kohn-Sham orbitals and the energy levels, Eᵥ, by the eigenvalues of the Kohn-Sham equation Hamiltonian. Therefore, the Kohn-Sham orbitals are computed by solving an equation that depends on the potential (the Kohn-Sham equation), and the potential depends on the charge density computed from the Kohn-Sham orbitals. In this way, again, a non-linear Poisson equation is solved (in which charge density depends on the potential).

In the approaches described above, electrons and holes are assumed to be in a state of thermal equilibrium with their environments. However, the non-linear Poisson equation can also be used to describe out-of-equilibrium situations. In such cases electron and hole densities can be calculated via other approaches. Examples include but are not limited to: non-equilibrium Green's functions combined with density functional theory (NEGF-DFT), with effective-mass theory (NEGF-EMT), or with k·p theory (NEGF-k·p).

Another broad category of charge density models is the ionization models. Indeed, there are potentially infinitely many ways in which the ionized donor and acceptor densities, N₊ and N₋ respectively, may be modelled in the calculation of the charge density. Here, two examples are detailed, bearing in mind that many others may be used.

A first one is the complete ionization model. Under complete ionization, it is simply assumed that all donors and acceptors are fully ionized, leading to

$${N}_{+} = {N}_{D} , {N}_{-} = {N}_{A} , \qquad ( 13 )$$

where N_{D} and N_{A} are the donor and acceptor density in the semiconductor, respectively. This approach is typically valid near room temperature or at very strong doping concentrations.

A second one is the incomplete ionization model. Under incomplete ionization, assuming thermal equilibrium of the dopants with the charge carriers, the ionized dopant concentrations are given by

$${N}_{+} = \frac{{N}_{D}}{1 + {g}_{D} exp [ ( {E}_{F} - {E}_{C} + {E}_{d} ) / {k}_{B} T ]} , \qquad ( 14 )$$

$${N}_{-} = \frac{{N}_{A}}{1 + {g}_{A} exp [ ( {E}_{V} - {E}_{F} + {E}_{α} ) / {k}_{B} T ]} , \qquad ( 15 )$$

where g_{D} (g_{A}) is the donor (acceptor) level degeneracy and E_{d} (Eₐ) is the donor (acceptor) binding energy.

Other categories exist. Charge-density models described above can be adapted to the case of semiconductor physics. Other non-linear equations of the same form as Eq. 1 also arise in several other contexts in which the charge density does not necessarily arise from electrons, holes, and ionized dopants. For example, a similar equation can be used to describe ionic solutions, in which charges arise from local equilibrium ion densities; in this context, the equation is known as the Poisson-Boltzmann equation.

Returning more generally to the computer-implemented method of solving a non-linear Poisson equation presented above in relationship with the flowchart of FIG. 1, it was found that though this method can be suitable for some embodiments, it may be less than suitable for other embodiments. In particular, it was found that non-convergence can be more and more likely as the temperature defined in the boundary conditions and/or variables of equations representing the physics problem decreases, to the point that this computer-implemented method can be considered unusable for some embodiments. This can particularly be the case when the physics problem defined by the boundary conditions represents quantum devices operating at cryogenic temperatures, such as below 100K, below 50K, below 10K, or below 1K, for instance, which are temperatures in which quantum electronic devices can be configured to operate. Accordingly, this latter computer-implemented method can be limited in terms of being suitable for use with a limited subset of physics problems and their associated boundary conditions.

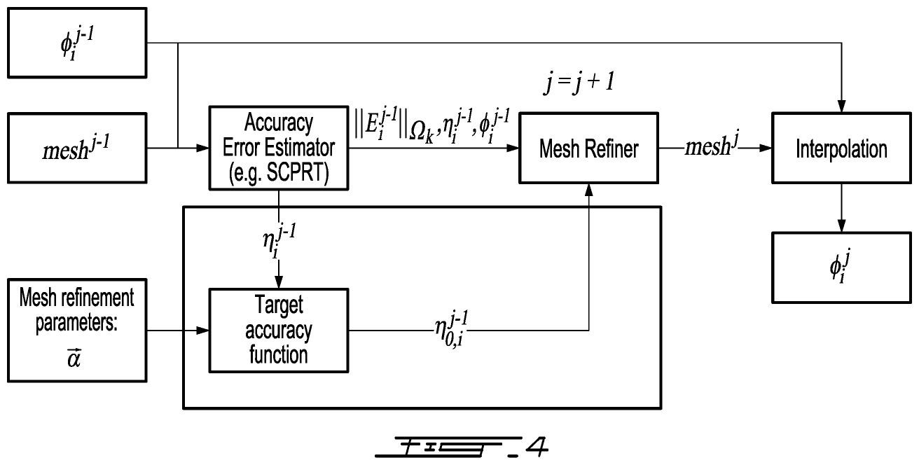

It was found that at least some of the limits of the latter computer-implemented method could be addressed or alleviated by an other computer-implemented method of solving a non-linear Poisson equation represented by the flowchart presented at FIG. 3. More specifically, it was found that this second computer-implemented method could lead to a reduced likelihood of non-convergence in some physics problems, and therefore be considered suitable for an at least partially non-overlapping subset of physics problems, i.e. reliably converge for at least some physics problems for which the method schematized in FIG. 1 would not reliably converge.

The computer-implemented method of solving a non-linear Poisson equation presented in FIG. 3 can be embodied similarly to the method presented above, with an exception that a mesh adapter module is also provided. In alternate embodiments to this embodiment as well, the positions of the charge density calculation module and of the linear solver module can be interchanged. In this embodiment, the mesh adapter module can be configured to adapt the mesh in an automated manner between two successive iterations. The mesh can also be adapted more than once over the entirety of the iterations.

In the specific embodiment presented in FIG. 3, an error analysis module is provided, and the mesh adapter module only adapts the mesh when the error analysis module determines that the error is not determined to be converging (e.g. diverges or saturates), though this is optional and in some embodiments, the mesh adapter module can operate at each iteration or a selection can be made to adapt the mesh or not based on other conditions than trends in the error of the current values (which, in an embodiment such as presented above, can be precision errors). In the illustrated embodiment, the value of a secondary counter j is also increased, and the mesh adapter module outputs a new mesh, mesh^{j} and a new potential, ϕᵢ^{j} defined based on that mesh.

In the next iteration performed by the charge density calculator module, the linear solver module, and the output comparator module, the values calculated in the previous iteration can be used as “starting” values (e.g. instead of an initial guess), and the method can be configured to solve electric potential and charge density for nodes of the newly adapted mesh. More specifically, in the illustrated embodiment, the charge density calculation module can use values of electric potential corresponding to the nodes of the modified mesh stemming from an interpolation based on the previously calculated values of charge density for the previous version of the mesh and the variations in the geometrical coordinates of the nodes, for instance.

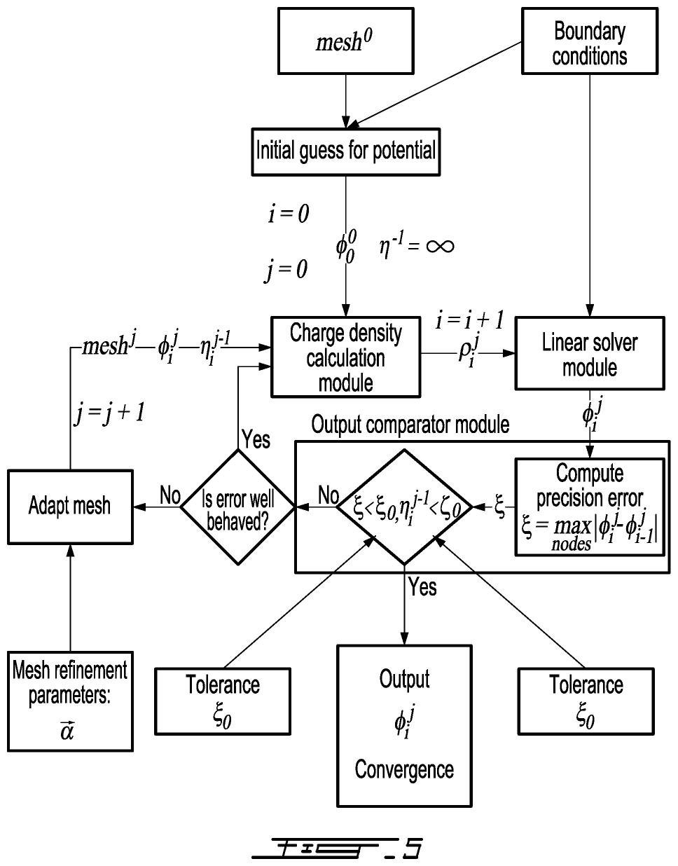

FIG. 4 presents a flowchart representing an example embodiment of a mesh adapter module in greater detail. The mesh adapter module can take as input the previous version of the mesh, mesh^{j-1}, and the current values outputted from the previous iteration which, in this embodiment where the charge density calculation module is used before the linear solver module, is the current values of the electric potential ϕᵢ^{j-1}. The current values could be current values of charge density in an embodiment where the linear solver module operates before the charge density calculation module for instance.

The mesh adapter module includes an accuracy error estimation function. This accuracy error estimation function can be used to estimate the accuracy error between the current values and the true solution values of the physical problem. The accuracy error estimation function can use the current values from the current iteration, and can thus yield values which are specific to that iteration. In the context where the mesh is a finite-element mesh, for instance, the accuracy error estimation function can be based on SCPRT for instance, though other types of accuracy error estimation functions can be used in other embodiments, and other types of accuracy error estimation functions can be used for other types of mesh, such as finite volume or finite difference meshes.

The mesh adapter module also has a mesh refiner function which is adapted to densifying the spatial distribution of the nodes in certain regions of the mesh. More specifically, the mesh refiner function can be adapted to identify regions of the mesh where the accuracy error is higher than in other regions, and densify the nodes in those regions. The densification of the nodes in those regions can be performed based on preset values, or can depend on the value of the accuracy error, such as by increasing the density more when the accuracy error is higher and increasing the density less when the accuracy error is lower.

In this embodiment, the mesh adapter module also has a target accuracy function. The target accuracy function can be configured for setting a target accuracy which is lower than the accuracy error which was estimated at the accuracy error estimator. The mesh refiner function can be configured to increase the density of the nodes in regions where the accuracy error is larger than the target accuracy error, for instance. The degree in which the target accuracy is set lower than the accuracy error can vary depending on the embodiment. For instance, in some embodiments, it may be preferred for the mesh adaptation function to be performed less aggressively, in which case the target accuracy can be set closer to the accuracy error, whereas in other embodiments, it may be preferred for the mesh adaptation function to be performed more aggressively, in which case the target accuracy can be set to be significantly lower than the accuracy error. Interestingly, the target accuracy can thus vary from one instance of the mesh refinement function to another over different iterations of the method, such, for instance, as a function of the specific accuracy error estimated for that instance of the function.

For the purpose of providing a full and clear description of one potential embodiment, a specific example in a context of a mesh provided in the form of a finite-element mesh, and where the potential, ϕᵢ^{j-1} is output by the linear solver module, will now be presented in detail. In this example, these inputs are passed through an error estimator function which estimates the energy norm of the error, ∥Eᵢ^{j-1}∥_{Ω}_{ₖ}, for each element, Ωₖ, of the mesh, mesh^{j-1}. Here, the elements of the mesh are indexed with k, and their volumes are denoted Ωₖ. In this example, the energy norm of a vector quantity v over some volume Ω is given by

$${‖ v ‖}_{Ω} = {\frac{1}{ε} [ {\int }_{Ω} dr {‖ v ‖}^{2} ]}^{1 / 2} , \qquad ( 16 )$$

where ∥⋅∥ is the L₂ norm. The error E^{j-1} is defined as

$${E}_{t}^{j - 1} = {\overline{σ}}_{i}^{j - 1} - {σ}_{i}^{j - 1} , \qquad ( 17 )$$

where σᵢ^{j-1}=ε∇ϕᵢ^{j-1} is the stress associated with ϕᵢ^{j-1} and σ̅ᵢ^{j-1} is an estimate of the true stress computed by the error estimator. One way to calculate σ̅^{j} is to use superconvergent patch recovery technique (SCPRT). However, other ways to estimate the error can be alternately used. For example, extensions of SCPRT exist. At this stage the estimated total relative error on the energy norm is computed

$${η}_{i}^{j - 1} = \frac{{‖ {E}_{i}^{j - 1} ‖}_{Ω}}{{‖ {σ}_{i}^{j - 1} ‖}_{Ω}} , \qquad ( 18 )$$

where Ω=∪ₖΩₖ. This estimated relative error, ηᵢ^{j-1}, is a measure of the inaccuracy of the solution ϕᵢ^{j-1} due to the finite nature of the mesh, mesh^{j-1}, that is being used to discretize the problem. In this embodiment, the approximate error, ηᵢ^{j-1}, is then passed to two separate functions: target accuracy (which can alternately be referred to here as Compute_eta in the specific context) and Mesh Refiner.
The Compute_eta function is one that has as inputs the estimated relative error, nᵢ^{j-1}, along with some mesh refinement parameters, a (which may be user defined for instance) and outputs a quantity η_{0,i}^{j-1} for the current mesh, mesh^{j-1}. The function Compute_eta can be any suitable function having an output, η_{0,i}^{j-1} which obeys

$$0 < {η}_{0 , i}^{j - 1} \leq {η}_{i}^{j - 1} . \qquad ( 19 )$$

A relatively simple example of such a function would be one where \vec{α} has a single entry 0≤α≤1 and Compute_eta computes η_{0,i}^{j-1} the following way

$${η}_{0 , i}^{j - 1} = α {η}_{i}^{j - 1} \qquad ( 20 )$$

The quantity η_{0,i}^{j-1} can be passed to the Mesh Refiner function along with ηᵢ^{j-1}, ∥Eᵢ^{j-1}∥_{Ω}_{ₖ}, and ϕᵢ^{j-1}. The Mesh Refiner function computes a new characteristic length at every node of the old mesh i.e. a size map for a new mesh, mesh^{j}. The mesh, mesh^{j}, will have elements distributed in such a way that the error, ∥Eᵢ^{j}∥_{Ω}_{ₖ} is uniform over k. In this embodiment, the potential ϕᵢ^{j-1} is interpolated over the new mesh, mesh^{j} to obtain ϕᵢ^{j}. Henceforth, rather than using mesh^{j-1} and ϕᵢ^{j-1}, the next iteration over the sequence of charge density calculation module, linear solver module, and output comparator module can use mesh^{j} and ϕᵢ^{j}. And the iterations can continue until convergence is met.

For the purpose of providing a detailed and complete description of one possible embodiment, additional information about the Compute_eta function will now be provided, though it will be understood that several variants may exist.

First, it will be stressed in this embodiment that the threshold η_{0,i}^{j} is dynamic and is computed for each pass based on ηᵢ^{j}. In this embodiment, the threshold η_{0,i}^{j} is thus not constant nor user-defined. Parameter, η_{0,i}^{j-1}, can be thought of as an indicator for how much the mesh, mesh^{j-1} is to be refined. The smaller η_{0,i}^{j-1} is, the more nodes mesh^{j} will be modified to have. Thus, in the example illustrated in Eq. 20, in the limit where a is very close to 1 (η_{0,i}^{j-1}˜ηᵢ^{j-1}) the mesh mesh^{j} will be very close to mesh^{j-1}. It will therefore take many more iterations of loop to get a mesh that converges. In contrast, in the limit where α is very close to 0 (η_{0,i}^{j-1}˜0) the mesh mesh^{j} will have many more nodes than mesh^{j-1}. In this case, the non-linear Poisson solver may converge very quickly in terms of iterations compared to the α˜1 case, but will converge on a mesh with many more nodes compared to the α˜1 case. There is thus a tradeoff between number of nodes of the final mesh and number of total iterations of the procedure: in the η_{0,i}^{j-1}˜ηᵢ^{j-1} case, more iterations may be required when compared to the η_{0,i}^{j-1}·0 case, however, the final mesh will also have less nodes.

Second, other mathematical functions, besides the one presented in Eq. 20, can be used to implement Compute_eta. In fact, any mathematical function could work provided Eq. 19 is respected. For example, a polynomial function can be used, where \vec{α} has 2 components, \vec{α}=(N,m) such that, N>1, and 0≤m≤1 and Compute_eta computes η_{0,i}^{j-1} in the following way:

$${η}_{0 , i}^{j - 1} = min ( {( {η}_{i}^{j - 1} )}^{N} , m ) . \qquad ( 21 )$$

If ηᵢ^{j-1}<1, then (ηᵢ^{j-1})^{N}<η^{j-1}. If not then 0<η_{0,i}^{j-1}=m<1, which respects Eq. 19.

Third, various approaches can be used for the mesh refiner, which may depend of the nature of the mesh, amongst others. A possible implementation of the mesh refiner module can be to generate characteristic lengths for a new mesh, mesh^{j}, such that its elements will have a uniform error, i.e. where ∥Eᵢ^{j}∥_{Ω}_{ₖ}, will be uniform over k. Such an approach would calculate the characteristic lengths for each element k of mesh mesh^{j}, hₖ^{j} via

$${h}_{k}^{j} = \frac{{h}_{k}^{j - 1}}{{χ}_{k}^{β + d / 2}} , \qquad ( 22 )$$

where β is the order of the mesh if k contains no singularity and β=λ if k contains a singularity of order λ, d is the dimension of the problem, and χₖ is a scaling factor for the characteristic lengths. This scaling factor is computed using

$${χ}_{k} = \frac{{‖ {E}_{i}^{j} ‖}_{{Ω}_{k}}}{{E}_{a}} , \qquad ( 23 )$$

$$where$$

$${E}_{a} = \frac{{η}_{0 , i}^{j - 1} {‖ {σ}_{i}^{j - 1} ‖}_{Ω}}{\sqrt{{M}_{e x p}}} \qquad ( 14 )$$

is the allowable error for each element. In Eq. 24, Mₑₓₚ is the expected number of elements of the mesh, mesh^{j}, and can be computed using

$${M}_{e x p} = {{M}_{j - 1} ( \frac{{η}_{i}^{j - 1}}{{η}_{0 , i}^{j - 1}} )}^{d / p} , \qquad ( 25 )$$

where p is the order of the mesh, and Mⱼ₋₁ is the number of nodes in the mesh, mesh^{j-1}.

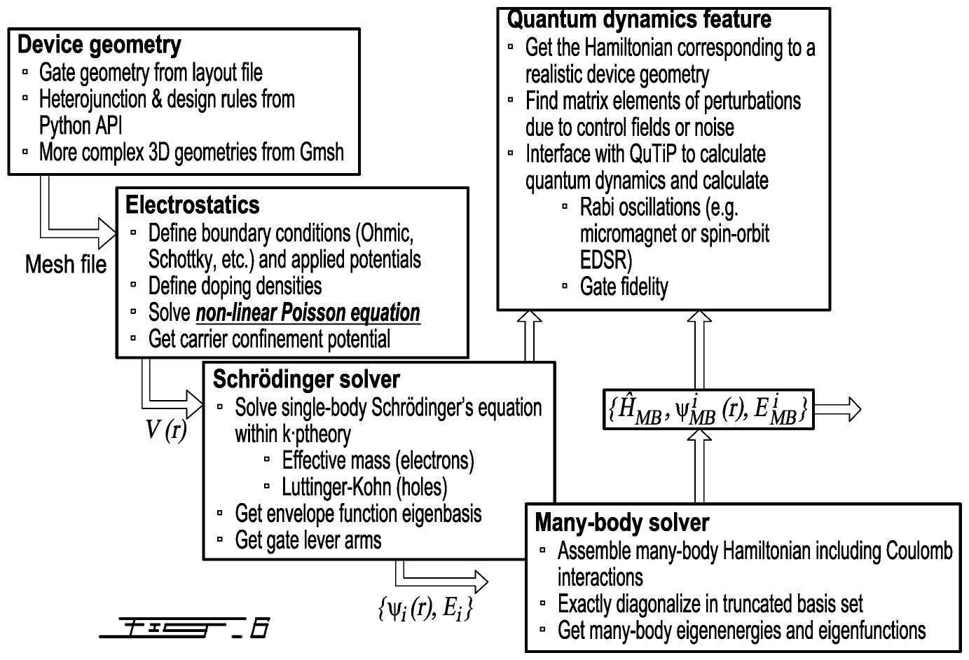

There are many possible ways to extend the non-linear adaptive Poisson algorithm that may further improve convergence. For example, one possible variant to the embodiment presented in FIG. 3 is presented in FIG. 5, which additionally uses a convergence criterion that checks whether ηᵢ^{j} is below some user-defined tolerance ζ₀. The main differences with the embodiment of FIG. 3 can be:
1. two user-defined tolerances as inputs (ξ₀ and ζ₀) and checks two conditions ξ<ξ₀, ηᵢ^{j-1}<ζ₀. instead of a single error check
2. at the start, set η₀⁻¹=∞. to force the second condition (ηᵢ^{j-1}<ζ₀) to fail before the mesh is refined.

Here, the loop keeps running until both ξ<ξ₀, ηᵢ^{j-1}<ζ₀ are satisfied. The quantity ηᵢ^{j} should be assumed to be independent of i, i.e. ηᵢ^{j}=η^{j}∀i. Since ηᵢ^{j-1} measures the accuracy of the solution (how close ϕᵢ^{j} is to the true value), converging in terms of both ξ₀ and ζ₀ (instead of just ξ₀) can lead to more accurate results.

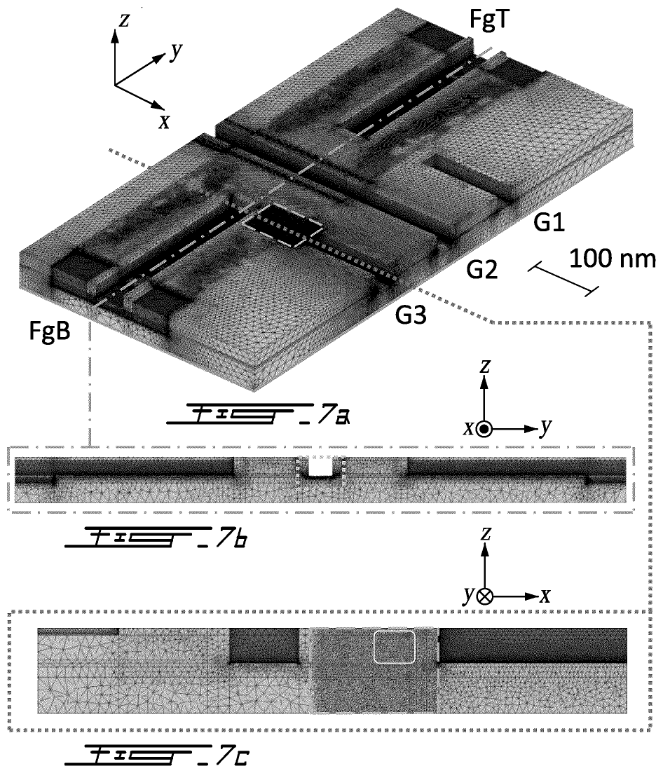

FIG. 6 presents an example of how a computer-implemented method of solving a non-linear poisson equation can be integrated more generally in a software package configured for simulating the behaviour of electronic devices, and more specifically here, quantum devices, and even more specifically, spin qubit devices operating at cryogenic temperatures. It will be understood that this example is but one of innumerable possible contexts in which a non-linear Poisson solver such as shown in FIG. 3 or 5, or otherwise as described above, can be implemented.

More specifically, silicon-based spin qubits are a promising candidate for the scalable implementation of quantum technologies because of their long coherence times and nanometer-scale dimensions enabling, in principle, to fit millions of qubits on a single chip. In addition, recent experiments have shown that high-quality spin qubits can be built with standard industrial microfabrication processes. High-yield and high-quality fabrication with excellent device uniformity can be expected to significantly accelerate the development of scalable silicon-based quantum computers by reducing prototype turnover time, increasing batch size, and thus generally shortening R&D cycles. As the technology matures and scales up, spin-qubit design and fabrication should follow the same workflow as conventional semiconductor technologies.

Apart from fabrication techniques, an essential component of the microelectronics engineering toolbox is technology computer-aided design software which is used to predict device performance and trends before fabrication. Even for seemingly simple tasks like the calculation of band diagrams, cryogenic temperatures (100 mK to 4 K) at which spin qubits are operated pose a significant challenge to solver convergence.

A more elaborated software solution referred to herein as Quantum-Technology Computer-Aided Design (QTCAD) including a 3D finite-element spin-qubit simulation package will now be presented. Among many other features, QTCAD includes non-linear Poisson and Schrödinger solvers enabling to calculate the electronic structure of gated quantum dot devices. These solvers were used to model a split-gate 28 nm Ultra-Thin Body and Buried (UTBB) oxide fully-depleted silicon-on-insulator (FD-SOI) device produced by standard STMicroelectronics fabrication processes. These simulations were used to interpret transport experiments performed at 1.4 K; in particular, they provided a quantitative understanding of channel activation and corroborated the experimental observation of side-gate-activated corner quantum dots. These simulations show robust convergence at an even lower temperature of 100 mK without user intervention thanks to QTCAD's adaptive meshing scheme, which can significantly relaxing the need for manual fine tuning of the mesh—a tedious and time-consuming step that often results in excessive mesh size and hampers seamless integration of simulation software into the gated quantum-dot design workflow.

At a mesoscopic scale, the conduction and valence band edges in a semiconductor device are determined by the electric potential φ that solves the non-linear Poisson equation

$$∇ \cdot ( ε ∇ φ ) = - q ( p - n + {N}_{+} - {N}_{-} ) , \qquad ( 26 )$$

where n, p, N₊, N₋ are the electron, hole, ionized donor, and ionized acceptor densities, respectively, E is the dielectric permittivity of the medium, and q is the elementary electric charge. Within the Thomas-Fermi approximation, and assuming a 3D isotropic and quadratic band model, carrier densities in electron and hole reservoirs are given by n=N_{c}F_{1/2}[(E_{F}−E_{C})/k_{B}T] and p=NᵥF_{1/2}[(E_{V}−E_{F})/k_{B}T], where E_{C(V)} is the conduction (valence) band edge, E_{F} is the Fermi level, T the temperature (assumed to be uniform), k_{B} the Boltzmann constant, and F_{1/2} the complete Fermi-Dirac integral of order ½. We have also introduced the electron (hole) effective density of states (DOS) N_{c(v)}=g_{c(v)}(2m_{c(v)}k_{B}T/πℏ²)^{3/2}/8, where ℏ is the reduced Planck's constant, m_{c(v)} is the electron (hole) DOS effective mass, and g_{c(v)} is the total electron (hole) band degeneracy, including both spin and valley degrees of freedom. The band edges are related to the electric potential through E_{c}=−q(φ−φ_{ref})−χ and E_{V}=E_{C}−E_{g}, where χ and E_{g} are the electron affinity and bandgap, respectively, and φ_{ref} is an arbitrary reference potential. At interfaces between materials, this approach automatically enforces Anderson's rule for band alignment.

Since carrier densities depend on electric potential, Eq. 26 is non-linear, mandating an iterative self-consistent approach. At cryogenic temperatures (below ˜100 K), Eq. 26 can be notoriously difficult to solve due to the Fermi-Dirac integrals which lead to fast variations of the carrier densities over very short length scales where the conduction or valence band edge crosses the Fermi level. To resolve this difficulty in a robust way, QTCAD implements a finite-element based non-linear Poisson solver able to account for arbitrary 3D first or second order meshes. In addition, an adaptive meshing algorithm automatically refines first-order meshes at problematic locations, leading to robust convergence at cryogenic temperature.

Focusing on conduction electrons within effective-mass theory, for a given confinement potential V(r)≡E_{C}, the single-electron energy levels E and eigenstates ψ(r) are given by the solution of the time-independent (steady-state) Schrödinger's equation

$$V ( r ) ψ ( r ) - \frac{{ℏ}^{2}}{2} ∇ \cdot [ {M}_{e}^{- 1} \cdot ∇ ψ ( r ) ] = E ψ ( r ) , \qquad ( 27 )$$

where Mₑ⁻¹ is the inverse effective mass tensor. Unlike Eq. 26, Eq. 27 is linear—no particular meshing scheme is required to converge to the solution of Schrödinger's equation.

In bulk silicon, the conduction band has six degenerate minima located at roughly 85% of the Brillouin zone boundary along the ±x, ±y, and ±z directions. Assuming [001] crystal orientation, under strong quantum confinement along z (e.g., in a two-dimensional gas), ±x and ±y valley eigenenergies lie several meV above ±z eigenenergies, because their effective mass along z is weaker. The remaining two-fold degeneracy is broken when the electron wavefunction overlaps with a sharp interface between two materials, e.g., at the interface between silicon and silicon dioxide. Therefore, in this work, we assume that the valley degeneracy is completely lifted when solving Schrödinger's equation, and use the inverse effective mass tensor corresponding to the ±z valleys: [[1/mₜ, 0,0], [0,1/mₜ, 0], [0,0,1/ [symbol] ]], where mₗ=0.916mₑ and mₜ=0.19mₑ are the longitudinal and transverse silicon effective masses, respectively, with mₑ the bare electron mass.

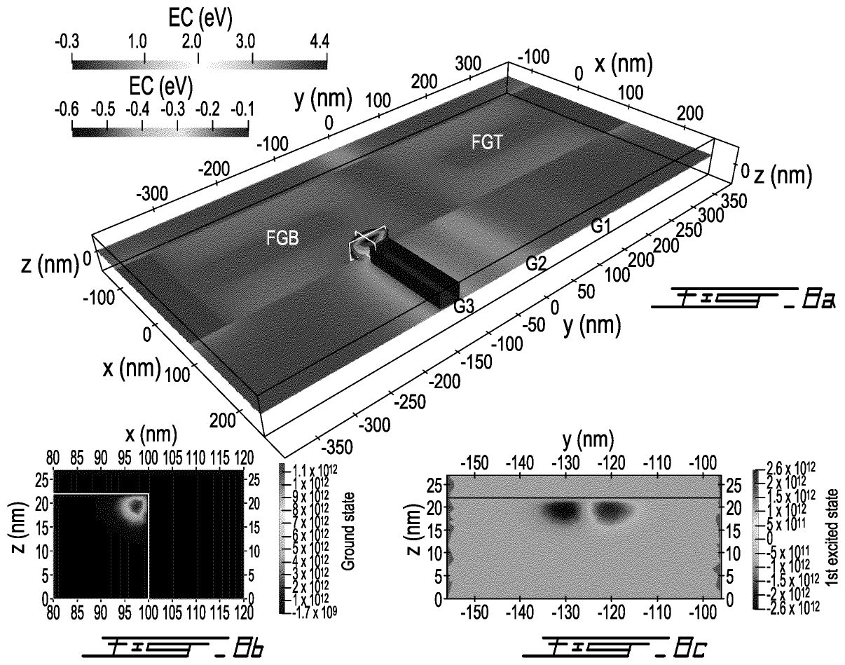

FIG. 7 illustrates the 3D CAD model employed to simulate the device (a more detailed description of 28 nm UTBB FD-SOI geometry is given in Ref. kriekouki2022interpretation). The source and drain are strongly n-doped, providing classical electron reservoirs that can be extended into the undoped silicon channel by applying positive biases on the bottom and top front gates (FgB and FgT, respectively). Side gates (G1, G2, and G3) control the formation of quantum dots in the channel. We will see that under sufficiently strong bias, G1 and G3 can create confinement potentials at corners of the channel.

Due to the large dimensions of the device, electrons are modelled everywhere as a 3D classical gas with density given by the Fermi-Dirac-integral expressions given below Eq. 26, with total degeneracy g_{c}=2×6=12 (2 for spin, 6 for valleys) and effective mass m_{c}=( [symbol] mₜ²)^{1/3}. A key exception however arises when G1 or G3 are sufficiently positively biased, in which case a region of strong quantum confinement is created (shown by the dashed pale blue lines in FIG. 7) in which we set the classical electron density to zero to model electrons quantum-mechanically. Because this device is unipolar and the bulk is excluded from the simulations, holes can be safely neglected in our calculation (p≈0).

When solving Poisson's equation, the following Dirichlet boundary conditions are assumed for each contact. In FIG. 7, the dark blue surfaces represent Ohmic boundary conditions, while the red surfaces represent metallic gate contacts, in which we assume a work function equal to the intrinsic silicon work function (flat band conditions). An additional Ohmic contact is assumed for the bottom surface of the simulation domain (magenta lines), which coincides physically with the interface between the buried oxide and weakly p-doped (10¹⁶ acceptors/cm³) bulk silicon.

More specifically, gate boundary conditions are applied at surfaces corresponding to metallic contacts G₁, G₂, G₃, FgB, and FgT (see FIG. 7). At gate boundaries, alignment of the metal, oxide, and semiconductor vacuum levels leads to an electric potential given by

$$φ = {φ}_{bias} + {φ}_{r e f} - {E}_{w} / q , \qquad ( 28 )$$

where φ_{bias} is the applied bias, φ_{ref} is the reference potential, E_{w} is the work function of the metallic gate, and q is the elementary positive electric charge.

Ohmic boundaries are applied at the source and drain contacts and at the interface between the buried oxide and the bulk (see FIG. 7).

At Ohmic boundaries, the semiconductor is assumed to be at a charge-neutral local equilibrium with its environment, leading to the condition

$$p - n + {N}_{+} - {N}_{-} = 0 , \qquad ( 29 )$$

where n(p) is the electron (hole) density and N₊(N₋) is the concentration of ionized donors (acceptors). Substituting the expressions of the equilibrium carrier densities, to find the boundary condition on the electric potential, we thus numerically solve the following non-linear equation for φ:

$${N}_{v} {F}_{1 / 2} [ - \frac{q ( φ - {φ}_{r e f} ) + χ + {E}_{g} + {E}_{F}}{{k}_{B} T} ] - {N}_{c} {F}_{\frac{1}{2}} [ \frac{q ( φ - {φ}_{r e f} ) + χ + {E}_{F}}{{k}_{B} T} ] + {N}_{+} - {N}_{-} = 0. \qquad ( 30 )$$

Importantly, in the CAD model employed here, we assume an idealized device with perfect materials. More realistic non-idealities like interface states, oxide charges, individual dopants, nuclear spins, valley coupling or surface roughness are left for future work.

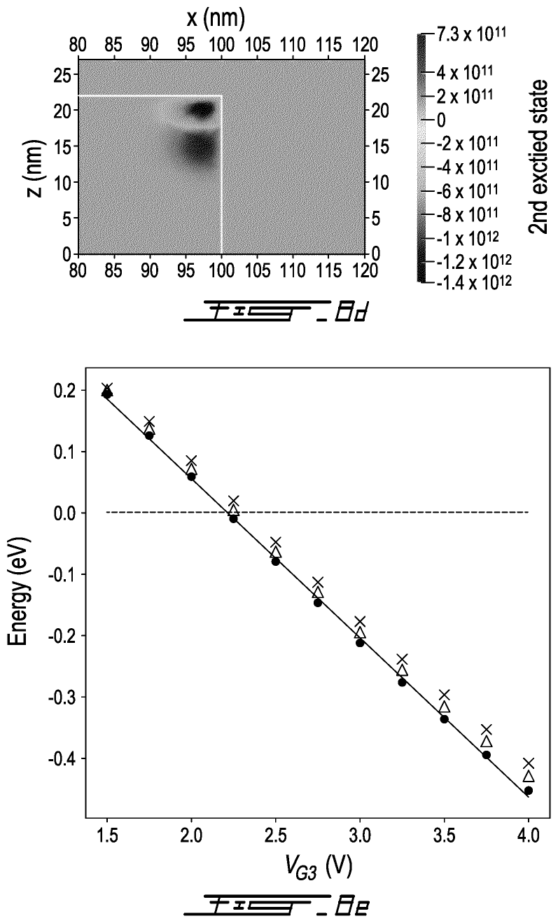

Under the assumptions and models described above, we solve the non-linear Poisson equation (Eq. 26) with the adaptive meshing technique using 8 cores of an AMD Ryzen Threadripper 3990X 64-Core Processor. At 100 mK, starting from a coarse uniform mesh containing 4,953 nodes, our algorithm iteratively refines the mesh until convergence (within a tolerance of 1 meV on φ) is finally achieved with the first-order mesh illustrated in FIG. 7, which contains 953,141 global nodes. In this process, we let the algorithm decide the mesh characteristic length everywhere except in the dot region illustrated by the dashed pale blue lines in FIG. 7, where we want to investigate formation of a quantum dot. In this region, we force the mesh characteristic length to 1.5 nm for improved accuracy of the Schrödinger solver. The resulting conduction band edge E_{c} is illustrated in FIG. 8(a). As shown by the isosurfaces (second color scale), a 3D potential well is formed in the corner of the silicon channel that faces gate G3. Due to mirror symmetry of the device along y, the same potential well may be formed in front of G1 by interchanging G1 and G3 biases.

To investigate the capability of the well to host bound states, we solve Schrödinger's equation [Eq. 27] inside the dot region for the first 10 energy levels and envelope functions. The first three envelope functions are illustrated in FIG. 8(b-d). While the lobes of the first excited state are oriented along the y axis, those of the second exited state are aligned with the z axis. These plots illustrate the 3D nature of quantum confinement in this FD-SOI device, and thus foster the need for 3D simulation tools like QTCAD for quantum-dot design. In such a 3D geometry, some common assumptions in the theoretical description of gated quantum dots, such as lifting of the valley degeneracy due to strong anisotropy of the quantum-confinement potential, may need to be revisited.

In FIG. 8(e) we show that the first three eigenenergies vary nearly linearly as a function of gate 3 bias. From a linear fit of the ground state energy, we find a lever arm of 0.26 eV/V of gate 3 on the energy levels of the dot, a quantity which plays a crucial role in the physics of charge stability diagrams,[8, 15] and coupling to microwave resonators.[7, 2, 19, 20] In addition, we find that the ground state crosses the Fermi level at V_{G3}≈2.2 V, which sets the bias that should be applied to load a single electron into the quantum dot, assuming that potential barriers surrounding the quantum dot are sufficiently low to allow transmission to and from the reservoirs.

To verify that convergence of the non-linear Poisson solver at 100 mK was not specific to the gate configuration employed here, we repeated the same calculation for 10 random bias configurations sampling a wide range of experimentally relevant voltages. In each case, convergence of φ within a tolerance of 1 meV was systematically achieved starting from the same coarse uniform initial mesh without any user intervention, and with the same solver parameters. Despite much lower temperature, final converged meshes contain between 570,786 and 1,025,089 nodes, a significant improvement compared with the 2,500,000-node mesh defined at 1.4 K. We have also successfully tested our adaptive mesh on completely different 3D geometries (e.g., a triangular fin field-effect transistor[5]) at similar temperatures (100 mK).

The non-linear Poisson equation is solved for the device illustrated in FIG. 7 of the main text for 10 random gate bias configurations. Apart from these gate biases, the conditions and parameters used for these simulations are all the same as those presented above. In particular, for consistency, we still define a quantum dot region with a fixed mesh characteristic length of 1.5 nm in front of G3, and in which classical charge density is set to zero. We also set V_{G1} to zero because, in the main text, we study the formation of a single quantum dot in front of G3.

The non-zero gate biases are sampled uniformly within the following ranges:
V_{FgB}∈[0,2]V, V_{FgT}∈[0,2]V,
V_{G2} ∈[−1,1]V, V_{G3} ∈[0,10]V,
V_{B} ∈[0,6]V.

This corresponds to typical bias values explored during experimental characterization of the device.

We find that the non-linear Poisson solver converges for each gate configuration after a handful of automatic mesh refinements (<10).

The computation times vary between 1,909 s and 7,711 s on 8 cores of an AMD Ryzen Threadripper 3990X 64-Core Processor. However, we observed that computation times can vary significantly depending on the number of users running calculations on this workstation. Therefore, the computation times listed here are only an indication of the order of magnitude of the time needed for a simulation.

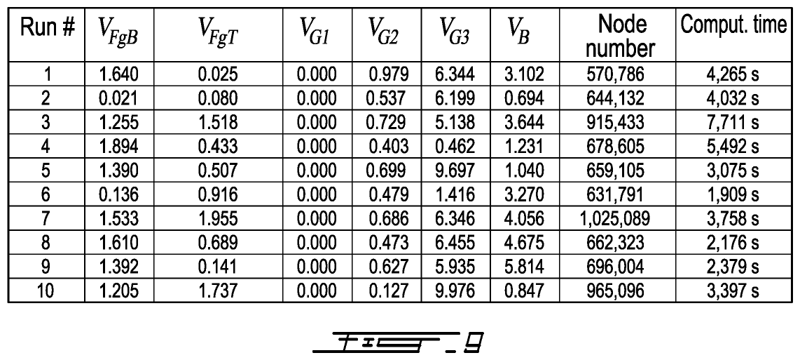

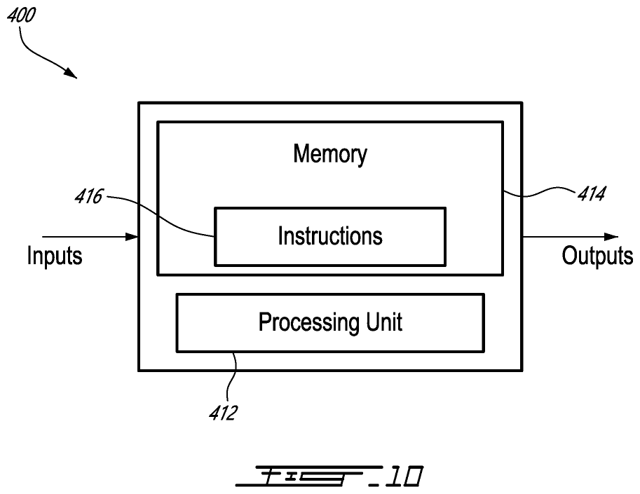

In addition, once the non-linear Poisson solver has converged on a given mesh, the same mesh can be used to achieve convergence for a slightly different gate bias configuration. Using the previous solution as an initial guess in the next bias configuration, convergence is then typically achieved in 10-20 iterations, i.e., an order of magnitude faster than for the results presented here. Furthermore, solving Schrödinger's equation over the dot region does not require generating a new mesh, and is typically done in less than a minute for the system presented here. The results are presented in a table as FIG. 9.

In conclusion, using QTCAD, we have achieved robust convergence of the 3D non-linear Poisson equation at 100 mK for a standard-process 28 nm UTBB FD-SOI structure, for a next-generation node of STMicroelectronics, and for triangular FinFET and gate-all around FET technologies. For an experimentally relevant bias configuration of the 28 nm device, we have clarified and illustrated the 3D geometry of quantum-confined states observed experimentally and studied theoretically in previous work. Robust convergence at cryogenic temperature prepares the ground for reliable and high-throughput technology computer-aided design of industrially fabricated spin-qubit devices. Future work will present QTCAD simulations of valley splitting, charge-stability diagrams, Coulomb interactions, and coherent spin-qubit control-key features to be addressed to predict experimentally relevant spin-qubit performance metrics before fabrication. Beyond spin qubits, this work opens the door to reliable TCAD simulations of cryogenically-cooled co-integrated qubit control electronics based on complementary metal-oxide-semiconductor (CMOS) technologies, a critical component for scalable quantum computing with spin qubits and superconducting qubits alike.

It will be understood that the expression “computer” as used herein is not to be interpreted in a limiting manner. It is rather used in a broad sense to generally refer to the combination of some form of one or more processing units and some form of memory system accessible by the processing unit(s). The memory system can be of the non-transitory type. The use of the expression “computer” in its singular form as used herein includes within its scope the combination of a two or more computers working collaboratively to perform a given function. Moreover, the expression “computer” as used herein includes within its scope the use of partial capabilities of a given processing unit. Example computers include desktop, laptop, supercomputers, etc.

A processing unit can be embodied in the form of a general-purpose micro-processor or microcontroller, a digital signal processing (DSP) processor, an integrated circuit, a field programmable gate array (FPGA), a reconfigurable processor, a programmable read-only memory (PROM), to name a few examples.

The memory system can include a suitable combination of any suitable type of computer-readable memory located either internally, externally, and accessible by the processor in a wired or wireless manner, either directly or over a network such as the Internet. A computer-readable memory can be embodied in the form of random-access memory (RAM), read-only memory (ROM), compact disc read-only memory (CDROM), electro-optical memory, magneto-optical memory, erasable programmable read-only memory (EPROM), and electrically-erasable programmable read-only memory (EEPROM), Ferroelectric RAM (FRAM) to name a few examples.

A computer can have one or more input/output (I/O) interface to allow communication with a human user and/or with another computer via an associated input, output, or input/output device such as a keybord, a mouse, a touchscreen, an antenna, a port, etc. Each I/O interface can enable the computer to communicate and/or exchange data with other components, to access and connect to network resources, to serve applications, and/or perform other computing applications by connecting to a network (or multiple networks) capable of carrying data including the Internet, Ethernet, plain old telephone service (POTS) line, public switch telephone network (PSTN), integrated services digital network (ISDN), digital subscriber line (DSL), coaxial cable, fiber optics, satellite, mobile, wireless (e.g. Wi-Fi, Bluetooth, WiMAX), SS7 signaling network, fixed line, local area network, wide area network, to name a few examples.

It will be understood that a computer can perform functions or processes via hardware or a combination of both hardware and software. For example, hardware can include logic gates included as part of a silicon chip of a processor. Software (e.g. application, process) can be in the form of data such as computer-readable instructions stored in a non-transitory computer-readable memory accessible by one or more processing units. With respect to a computer or a processing unit, the expression “configured to” relates to the presence of hardware or a combination of hardware and software which is operable to perform the associated functions. Different elements of a computer, such as processor and/or memory, can be local, or in part or in whole remote and/or distributed and/or virtual.

The methods and systems of the present disclosure may be implemented in a high level procedural or object oriented programming or scripting language, or a combination thereof, to communicate with or assist in the operation of a computer. Alternatively, the methods and systems described herein may be implemented in assembly or machine language. The language may be a compiled or interpreted language. Program code for implementing the methods and systems described herein may be stored on a non-transitory storage media or a device, for example a ROM, a magnetic disk, an optical disc, a flash drive, or any other suitable storage media or device. The program code may be readable by a general or special-purpose programmable computer for configuring and operating the computer when the storage media or device is read by the computer to perform the procedures described herein. Embodiments of the methods and systems described herein may also be considered to be implemented by way of a non-transitory computer-readable storage medium having a computer program stored thereon. The computer program may comprise computer-readable instructions which cause a computer, or more specifically the processing unit 402 of the computing device 400, to operate in a specific and predefined manner to perform the functions described herein, for example those described in the method 500.

Computer-executable instructions may be in many forms, including program modules, executed by one or more computers or other devices. Generally, program modules include routines, programs, objects, components, data structures, etc., that perform particular tasks or implement particular abstract data types. Example of program modules are the modules and functions referred to above Typically the functionality of the program modules may be combined or distributed as desired in various embodiments. The technical solution of embodiments may be in the form of a software product. The software product may be stored in a non-volatile or non-transitory storage medium, which can be a compact disk read-only memory (CD-ROM), a USB flash disk, or a removable hard disk. The software product includes a number of instructions that enable a computer device (personal computer, server, or network device) to execute the methods provided by the embodiments.

The embodiments described herein are implemented by physical computer hardware, including computing devices, servers, receivers, transmitters, processors, memory, displays, and networks. The embodiments described herein provide useful physical machines and particularly configured computer hardware arrangements. The embodiments described herein are directed to electronic machines and methods implemented by electronic machines adapted for processing and transforming electromagnetic signals which represent various types of information. The embodiments described herein pervasively and integrally relate to machines, and their uses; and the embodiments described herein have no meaning or practical applicability outside their use with computer hardware, machines, and various hardware components. Substituting the physical hardware particularly configured to implement various acts for non-physical hardware, using mental steps for example, may substantially affect the way the embodiments work. Such computer hardware limitations are clearly essential elements of the embodiments described herein, and they cannot be omitted or substituted for mental means without having a material effect on the operation and structure of the embodiments described herein. The computer hardware is essential to implement the various embodiments described herein and is not merely used to perform steps expeditiously and in an efficient manner.

As can be understood, the examples described above and illustrated are intended to be exemplary only. The scope is indicated by the appended claims.

What is claimed is:

1. A computer-implemented method of simulating the behaviour of an electronic device in the context of given boundary conditions, the behavior including a charge density distribution and an electric potential distribution over a geometry of the electronic device, the method comprising:
providing a mesh having nodes spatially distributed across a geometrical model of the electronic device;
calculating, taking into consideration the boundary conditions imposed to some of the nodes, and for each of the remaining ones of the nodes, i) current values of electric potential using a linearization of a non-linear Poisson equation, and ii) current values of charge density using a charge density model, wherein the current values of a first one of electric potential and of charge density are calculated further based on starting values of a second one of electric potential and of charge density, and the current values of the second one is calculated further based on the current values of the first one;
determining an error for the current values;
when the error is not within an error threshold, changing the spatial distribution of the nodes to a current spatial distribution of nodes, and repeating said steps of calculating and determining i) using the current values previously calculated as the starting values and ii) using the current spatial distribution of nodes for the nodes; and
when the error is within the error threshold, outputting the current values of electric potential and charge density as a simulated behaviour of the electronic device.

2. The computer-implemented method of claim 1 wherein said calculating includes calculating the current values of charge density first, based on starting values of electric potential, and subsequently calculating the current values of the electric potential, using the calculated current values of charge density.

3. The computer-implemented method of claim 1 wherein said determining an error for the current values includes determining a precision error between the starting values and the current values.

4. The computer-implemented method of claim 1 further comprising determining an accuracy error for the current values, wherein the step of changing the spatial distribution of the nodes includes densifying the spatial distribution of the nodes in regions having a larger accuracy error.

5. The computer-implemented method of claim 4 wherein the step of changing the spatial distribution of the nodes includes determining a target accuracy error which is lower than the accuracy error, and densifying the spatial distribution of the nodes in regions having a larger accuracy error compared to the target accuracy error.

6. The computer-implemented method of claim 5 wherein the mesh is a finite element mesh, the accuracy error is an accuracy error for elements of the mesh, determining the accuracy error includes using SCPRT, and determining the target accuracy error includes computing eta.

7. The computer-implemented method of claim 1 wherein, when the precision error is not within the precision error threshold, determining whether the precision error is converging or not converging, and wherein the steps of changing the spatial distribution of the nodes is contingent upon determining that the precision error is not converging.

8. A computer implemented method of determining the behaviour of an electronic device in predetermined conditions, the behavior including a charge density distribution and an electric potential distribution over a geometry of the electronic device, the method comprising:
providing an initial mesh having nodes distributed across a geometric model of the electronic device, each node having an initial spatial coordinates corresponding to a location forming part of the geometric model;
providing a charge density model;
provide a non-linear Poisson equation of the form ∇(ε∇ϕ)=−ρ, where ε is the dielectric permittivity of each one of the nodes, ϕ is the electric potential of each one of the nodes, and ρ is the charge density of each one of the nodes, and where both the electric potential and the charge density are i) both a priori unknown and ii) related to one another based on the charge density model;
providing a linearization of the non-linear Poisson equation;
providing boundary conditions having set values of charge density and electric potential for some of the nodes;
providing an initial values of a first one of electric potential and charge density for remaining ones of the nodes;
providing a precision error threshold;
calculating a values of the second one of electric potential and charge density for the remaining ones of the nodes;
calculating a subsequent values of the first one of electric potential and charge density for remaining ones of the nodes;
wherein said calculating values for charge density includes using the charge density model, and calculating values for the electric potential uses the linearization of the non-linear Poisson equation;
calculate a precision error between the initial values and the subsequent values;
when the precision error is within the threshold, outputting the latest calculated values of electric potential and charge density; and
when the precision error is not within the threshold, generating a subsequent mesh with a different spatial distribution of nodes than the distribution of nodes of the initial mesh, and repeating the steps of calculating using the latest calculated value of electric potential or charge density as the initial value of electric potential and charge density.

9. A computer-implemented method of simulating the behaviour of a composition of matter in the context of given boundary conditions, the behavior including a charge density distribution and an electric potential distribution over a geometry of the composition of matter, the method comprising:
providing a mesh having nodes spatially distributed across a geometrical model of the composition of matter;
calculating, taking into consideration the boundary conditions imposed to some of the nodes, and for each of the remaining ones of the nodes, i) current values of electric potential using a linearization of a non-linear Poisson equation, and ii) current values of charge density using a charge density model, wherein the current values of a first one of electric potential and of charge density are calculated further based on starting values of a second one of electric potential and of charge density, and the current values of the second one is calculated further based on the current values of the first one;
determining a precision error between the starting values and the current values;
when the precision error is not within a precision error threshold, changing the spatial distribution of the nodes to a current spatial distribution of nodes, and repeating said steps of calculating and determining i) using the current values previously calculated as the starting values and ii) using the current spatial distribution of nodes for the nodes; and
when the precision error is within an precision error threshold, outputting the current values of electric potential and charge density as a simulated behaviour of the composition of matter.
